(19) United States
(12) Patent Application Publication
(10) Pub. No.: US 2021/0212349 A1
(43) Pub. Date: Jul. 15, 2021

(54) Consumable Product Comprising Malted Dehulled Oats

(71) Applicant: Lantmännen Functional Foods AB, Stockholm (SE)

(72) Inventors: Ivar Lönnroth, Mölndal (SE); Lin Shi, Xi'an (CN); Rikard Landberg, Göteborg (SE)

(73) Assignee: Lantmännen Functional Foods AB, Stockholm (SE)

(21) Appl. No.: 17/193,092

(22) Filed: Mar. 5, 2021

Related U.S. Application Data
(63) Continuation of application No. PCT/EP2019/076394, filed on Sep. 30, 2019.

(30) Foreign Application Priority Data
Sep. 28, 2018 (SE) 1851168-3

(51) Int. Cl. A23L 7/25; A23L 29/00; A23L 7/143; A61K 36/899; A61K 31/192

(52) U.S. Cl. CPC A23L 7/25; A23L 29/045; A61K 31/192; A61K 36/899; A23L 7/143

(57) ABSTRACT

The disclosure relates to a consumable product comprising malted dehulled oats and/or a leachate of malted dehulled oat, wherein said consumable product induces endogenous production of antisecretory factor (AF) protein and/or fragments thereof in a subject after consumption. The malted dehulled oats comprised in the consumable product disclosed herein is produced by a novel malting process.

The malted dehulled oats and/or a leachate of malted dehulled oats comprised in the consumable product comprises (i) avenanthramide D, wherein the concentration of (i) is higher as compared to the corresponding non-malted dehulled oats, and optionally one or more of the compounds selected from the group consisting of (ii) avenanthramide A, (iii) avenathramide C, (iv) avenanthramide C methyl ester, (v) (Z)-N-feruloyl 5-hydroxyanthranilic acid, (vi) avenanthramide G, and (vii) a compound selected from the group consisting of guaiacol or a derivative thereof, L-tryptophan , DL-phenylalanine, and any combination thereof, wherein the concentration of one or more of (ii-vii) is higher as compared to in the corresponding non-malted dehulled oats.

The disclosure further provides use of the consumable product as food or feed for humans and/or animals, as well as for medical use.

### TECHNICAL FIELD

The present disclosure relates to a consumable product comprising malted dehulled oats and/or a leachate of said malted dehulled oats, wherein said consumable product induces endogenous production of antisecretory factor (AF) protein and/or fragments thereof in a subject after consumption. The malted dehulled oats of the consumable product comprise (i) avenanthramide D at a concentration which is substantially higher as compared to in the corresponding non-malted dehulled oats.

The consumable product according to the present disclosure comprises malted dehulled oats and/or leachate of said malted dehulled oats which is obtained from a novel malting process comprising the steps of dehulling oats kernels, malting said dehulled oats kernels at a very low temperature from about 5° C. to about 20° C., and subsequently drying said dehulled oats kernels at no more than 80° C. air temperature.

The present disclosure further relates to a consumable product comprising and/or consisting of malted dehulled oats and/or a leachate of said malted dehulled oats produced in accordance with the herein described malting process, which comprises malted dehulled oats and/or leachate of malted dehulled oats in an amount sufficient to increase the amount of antisecretory factor (AF) protein and/or fragments thereof in the subject's blood to at least about 0.7, such as at least 1 Units/ml blood, and to the use of the consumable product as food or feed and/or supplement to food or feed for humans and/or animals.

### BACKGROUND

### Antisecretory Factor (AF) Protein

The antisecretory factor (AF) is a class of proteins that occurs naturally in the body. Antisecretory factor (AF) protein is a 41 kDa protein that was originally described to provide protection against diarrhoea diseases and intestinal inflammation (for a review, see Lange and Lönnroth, 2001). The antisecretory factor (AF) protein has long since been sequenced and its cDNA cloned (see SEQ ID NO: 1). The antisecretory activity seems to be mainly exerted by a peptide located between the amino acid positions 35 and 50 on the antisecretory factor (AF) protein sequence which comprises at least 4-16, such as 4, 6, 7, 8 or 16 amino acids of the consensus sequence. The biological effect of AF is exerted by any peptide or polypeptide comprising at least 6 amino acids as shown in SEQ ID NO: 2 (AF-6), of said consensus sequence, or a modification thereof not altering the function of the polypeptide and/or peptide, such as by a peptide as shown in SEQ ID NO: 3 (AF-16), or in SEQ ID NO: 4 (AF-8).

It has been shown that the antisecretory factor (AF) protein is to some extent homologous with the protein S5a, and Rpn10, which constitutes a subunit of a constituent prevailing in all cells, the 26 S proteasome, more specifically in the 19 S/PA 700 cap. In the present disclosure, antisecretory factor (AF) proteins are defined as a class of homologue proteins having the same functional properties. Antisecretory factor (AF) protein is also highly similar to angiocidin, another protein isoform known to bind to thrombospondin-1 and associated with cancer progression.

Immunochemical and immunohistochemical investigations have revealed that the antisecretory factor (AF) protein is present and may also be synthesized by most tissues and organs in a body.

Synthetic peptides, comprising the antidiarrheal sequence, have prior been characterized (see WO 97/08202; WO 05/030246; WO 2007/126364; WO 2018/015379).

Antisecretory factor (AF) proteins and peptides have previously been disclosed to normalize pathological fluid transport and/or inflammatory reactions, such as in the intestine and in the central nervous system after challenge with the cholera toxin (WO 97/08202). WO 97/08202 discloses structures of certain antisecretory proteins, and their active parts are characterized. A synthetic ASP prepared by recombinant genetic engineering or by solid phase technology and having definite structures has been shown to have a general controlling influence on the body fluid flow over living cell membranes.

Food and feed with the capacity to either induce endogenous synthesis of AF or uptake of added AF have therefore been suggested to be useful for the treatment of oedema, diarrhoea, dehydration and inflammation in WO 97/08202. WO 98/21978 discloses the use of products having enzymatic activity for the production of a food that induces the formation of antisecretory factor (AF) proteins after consumption. WO 00/038535 further discloses food products enriched and/or naturally rich in native antisecretory factor (AF) proteins as such.

Antisecretory factor (AF) proteins and fragments thereof have also been shown to improve the repair of nervous tissue, and proliferation, apoptosis, differentiation, and/or migration of stem and progenitor cells and cells derived thereof in the treatment of conditions associated with loss and/or gain of cells (WO 05/030246) and to be equally effective in the treatment and/or prevention of intraocular hypertension (WO 07/126364), as for the treatment and/or prevention of compartment syndrome (WO 07/126363).

From the Swedish Patent SE 9000028-2 (publication No. 466,331) it is known that the formation of an antisecretory factor (AF) or an antisecretory factor (AF) protein (in SE 9000028-2 named ASP: also named FIL) can be stimulated by adding, to the animals' feed, certain sugars, amino acids and amides. The kinds and amounts of these substances to be used for the formation of an interesting amount of ASP is determined by a method disclosed in the patent. Briefly, this method involves measurement of a standardized secretion response in the small intestine of rat. From the patent it is evident that the induced ASPs formed direct the secretion of body fluid into the intestine. In said patent, the content or amount of natural antisecretory proteins is defined by its effect on the fluid secretion into the small intestine of laboratory rats having been challenged with cholera toxin (RTT-test). One ASP Unit (FIL Unit) corresponds to a 50% reduction of the fluid flow in the rat's intestine compared to a control without induced ASP. The antisecretory proteins are active in extremely small amounts and, therefore, it is often easier to determine them by their effect than by their mass.

From WO 98/21978 it is known that the formation of ASP can be induced in the body by consumption of a certain kind of food having enzymatic activity. The effect of the induction and, owing to that, the formation of ASP varies according to the individual and its symptoms and takes place with a strength and induction period unpredictable so far. However, they can be measured afterwards, and necessary corrections can be made with the guidance of said measurements. It is mentioned that the products may be malted cereals such as malted oats.

### Avenanthram Ides

Avenanthramides are a group of phenolic compounds comprising substituted N-cinnamoylanthranilic acids derived from cinnamic acid or a derivative thereof and anthranilic acid or a derivative thereof. The avenanthramides are mainly found in oats and have been reported to impart properties such as anti-inflammatory properties, antioxidant properties and anti-itch properties. In oat, the most abundant avenanthramides have been reported to be avenanthramides A, B, C, O, P and Q also called avenanthramides 2p, 2f, 2c, 2p_{d} and 2c_{d} as shown herein. The former nomenclature using capital letters is called Collin's nomenclature while the latter nomenclature is called Dimberg's modified nomenclature. In Dimberg's nomenclature the number refers to the anthranilic acid or a derivative thereof and the letter refers to the cinnamic acid or derivative thereof. For instance, “2” refers to 5-hydroxyanthranilic acid and “p” refers to p-coumaric acid. In addition, the letter “d” stands for double bond. In an example, avenanthramide A (2p) differs from avenanthramide 0 (2p_{d}) in the number of double bonds as shown in Scheme 1 below.

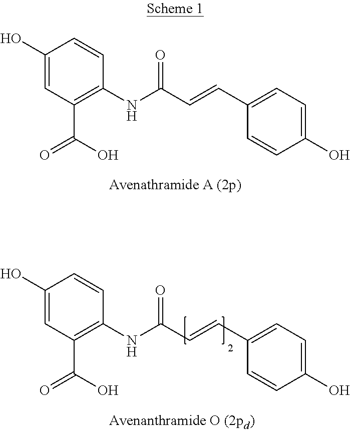

The report “A study of avenanthramides in oats for future applications” by Eléne Karlberg, Uppsala University School of Engineering, published in June 2010, discloses a method for enrichment of avenanthramides involving steeping and germination of oats at low pH. It is stated that an oats extract containing oats material subjected to this method would comprise positive physiological effects caused by avenanthramides and also beneficial effects originating from β-glucan.

WO 2010/108277 discloses methods for increasing the levels of avenanthramides in oats through false malting. Oats are first subject to induction or enhancement of a secondary dormancy, and then malted for up to 5 days at an elevated temperature. The malted but not germinated oats are then dried and used as is, or further processed or milled to produce food, feed, nutraceutical or personal care products and ingredients.

WO 2015/179676 discloses a composition and method for an avenanthramide-enriched, oat-based product having improved health effects. The oat-based product includes an avenanthramide ingredient having avenanthramides 2c:2p:2f in ratios comprising at least one of 1:1:1 or 1:2:2. The avenanthramide ingredient may be derived synthetically or recovered from processing raw oats into constituent oats fractions.

WO 2007/52153 states that it is known that the concentration of avenanthramides increase in the oats' endosperm upon steeping in water. It is also stated that it has been reported that avenanthramides are thermally stable to steam processing, and that these studies may suggest that malting oats may contribute to increased antioxidant properties due to elevated levels of avenanthramides but that the role of malting to increase the antioxidant properties of oats has not been reported in the scientific literature.

It has also been reported that oats may comprise or be mixed with the amino acid tryptophan.

U.S. Pat. No. 4,581,847 discloses novel plant genotypes, and in particular novel genotypes of cereal crops, including maize, rice, wheat, barley, sorghum, oats, rye, and millet, which produce increased levels of free tryptophan.

WO 2007/117815 discloses non-heat treated high amino acid feed and the dry milling process used to produce the feed and ethanol. In particular, it is disclosed a high amino acid feed having highly digestible proteins including amino acid residues substantially free of thermal input related damage. The feed may be produced from seed such as oats. The amino acids may comprise tryptophan.

WO 2017/09004 discloses a process for producing egg yolk with high content of AF-16. The process involves feeding a poultry, such as a hen, an AF-16 inducing pelleted feed for poultry comprising at least 0.14% free tryptophan, or at least 1-2 g tryptophan/kg feed, and thereafter harvesting egg from said poultry, separating egg yolk from egg white, and alternatively spray-drying, fluid-bed drying, grinding, leaching, extracting, evaporating, membrane filtrating, and/or or freeze-drying said egg yolk.

It is an object of the present disclosure to provide a consumable product such as food, feed and/or food- or feed-supplement comprising compounds such as phenolic acids and/or avenanthramides which stimulate and/or induce endogenous production of antisecretory factor (AF) protein, peptides and/or fragments thereof in a subject, such as a human or an animal, after consumption.

It is an object of the present disclosure to provide such a consumable product wherein the stimulating and/or inducing compounds are provided in malted dehulled oats.

Further, it is an object of the present disclosure to overcome or at least alleviate some of the disadvantages of known malting processes for producing food products comprising compounds such as phenolic acids and/or avenanthramides with health improving effects.

### SUMMARY

The present disclosure provides a consumable product comprising malted dehulled oats and/or a leachate of said malted dehulled oats comprising:

(i) avenanthramide D,

wherein the concentration of (i) is higher as compared to the corresponding non-malted dehulled oats, and

wherein the consumable product induces endogenous production of antisecretory factor

(AF) protein and/or fragments thereof in a subject after consumption.

The malted dehulled oats may further comprise one or more of:

(ii) avenanthramide A,

(iii) avenanthramide C,

(iv) avenanthramide C methyl ester,

(v) (z)-N-feruloyl 5-hydroxyanthranilic acid, and optionally

(vi) avenanthramide G,

wherein the concentration of one or more of (ii), (iii), (iv), (v) and (vi) is higher as compared to tin he corresponding non-malted dehulled oats.

The malted dehulled oats may also comprise:

(vii) a compound selected from the group consisting of guaiacol or a derivative thereof, L-tryptophan, DL-phenylalanine, and any combination thereof,

wherein the concentration of one or more of (vii) is higher as compared to in the corresponding non-malted dehulled oats. The guaiacol derivative may be ferulic acid, sinapic acid and/or p-coumaric acid.

A consumable product disclosed herein induces endogenous production of antisecretory factor (AF) protein and/or fragments thereof in a subject after consumption. The extent of the induction of said endogenous production of the antisecretory factor (AF) protein and/or fragments thereof may be adjusted by providing an appropriate amount of the consumable product to a subject in need thereof.

Consequently, the consumable product of the present invention may be used in the treatment, prevention and/or prophylaxis of an abnormal physiological condition characterized by and/or associated with elevated and/or pathologically high levels of body fluid discharge. Further, the consumable product of the present invention may be used in a treatment and/or prevention of a condition responsive to increased levels of antisecretory factor protein and/or antisecretory protein fragments in the blood of a patient. For instance, the consumable product may be used to treat diarrhoea, oedema and/or conditions involving inflammation in a subject such as a human and/or an animal. In a further example, the condition to be treated with the consumable product described herein may be selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, compartment syndrome, lipid raft disfunction, Alzheimer's disease, Parkinson's disease, encephalitis, and Meniere's disease.

The consumable product may comprise malted dehulled oats and/or leachate of malted dehulled oats in an amount sufficient to increase the amount of antisecretory protein and/or fragments thereof in the subject's blood to at least 1 units/ml.

In particular, the present disclosure provides a consumable product comprising malted dehulled oats and/or a leachate of said malted dehulled oats, wherein said malted dehulled oats are produced by a malting process characterized by comprising the steps of:
a. dehulling oats kernels,
b. wet steeping of the dehulled oats kernels at a temperature from 5° C. to 20° C.
c. germinating/growing of said dehulled oats kernels at a temperature from 5° C. to 20° C.,
d. optionally repeating any one of steps b-c, and subsequent
e. drying of said dehulled oats kernels at no more than 80° C. air temperature.

Optionally, the steeped kernels of step b. can be dried before germination. In the present context, the terms “germinating” and “growing” are interchangeable.

The malted dehulled oats produced by said novel malting process comprise avenanthramide D at a higher concentration as compared to the corresponding non-malted dehulled oats and induce endogenous production of antisecretory factor (AF) protein and/or fragments thereof in a subject after consumption.

A consumable product is so disclosed, produced by a malting process according to the present invention, wherein the wet steeping of the dehulled oats kernels in step a. is performed at a temperature from 7° C. to15° C. for 1-3 days, such as at a temperature of no more than 15° C. for at least 26 hours.

A consumable product is disclosed produced by a malting process according to the present invention, wherein the germinating of said dehulled oats kernels in step d. is performed for 5-9 days at a temperature of 12° C. to 15° C., such as for 7-9 days at a temperature of 12° C. to 15° C., such as for 9 days at a temperature not exceeding 12° C. and/or for 7 days at a temperature not exceeding 15° C.

The malted dehulled oats of the present invention typically comprise:

(i) avenanthramide D, wherein the concentration of (i) is at least 50%, such as at least 100%, 150%, 200%, 250%, 300%, 350%, 400%, 450%, or 500% higher as compared to in the corresponding non-malted dehulled oats.

The malted dehulled oats of the present invention can further comprise one or more of:

(ii) avenanthramide A,

(iii) avenathramide C,

(iv) avenanthramide C methyl ester,

(v) (Z)-N-feruloyl 5-hydroxyanthranilic acid, and optionally
(vi) avenanthramide G, and

wherein the concentration of one or more of (ii), (iii), (iv), (v) and (vi) is higher as compared to in the corresponding non-malted dehulled oats.

The malted dehulled oats of the present invention typically comprise: one or more of (ii), (iii), (iv), (v) and (vi), wherein the concentration of one or more of (ii), (iii), (iv), (v) and (vi) is at least 50%, such as at least 100%, 150%, 200%, 250%, 300%, 350%, 400%, 450%, or 500% higher as compared to in the corresponding non-malted dehulled oats.

The malted dehulled oats of the present invention can further again comprise: (vii) a compound selected from the group consisting of guaiacol or a derivative thereof, L-tryptophan, DL-phenylalanine, and any combination thereof, wherein the concentration of one or more of (vii) is higher as compared to in the corresponding non-malted dehulled oats.

The guaiacol derivative elevated in the malted dehulled oats of the present invention can be ferulic acid, sinapic acid and/or p-coumaric acid.

A consumable product according to the present invention, comprises malted dehulled oats and/or a leachate of said malted dehulled oats in an amount sufficient to increase the amount of antisecretory protein and/or fragments thereof in the subject's blood to at least about 1 unit/ml.

In one embodiment, a consumable product according to the present invention consists of malted dehulled oats and/or a leachate of said malted dehulled oats having been malted with the novel malting process disclosed herein.

A consumable product according to the present invention can be a food, feed, a food supplement and/or a nutraceutical, for human and/or animal consumption. It can be a feed for animals such as poultry and/or livestock animals. It can be in the form of a liquid, a solid or a combination thereof.

A consumable product disclosed herein has antisecretory properties, anti-diarrhoeal properties and/or anti-inflammatory properties.

In particular, a consumable product according to the present invention can be a functional food product and/or a pharmaceutical product for use as a medicament.

A consumable product according to the present invention can be for use in treatment, prevention, amelioration and/or prophylaxis of an abnormal physiological condition caused by pathologically high levels of body fluid discharge, such as for use in the treatment of a condition responsive to increase of levels of antisecretory factor protein and/or antisecretory protein fragments in the blood of a patient, wherein said condition can be selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

A consumable product according to the present invention can also be for use in the preparation of a pharmaceutical composition for use in treatment, prevention, amelioration and/or prophylaxis of an abnormal physiological condition caused by pathologically high levels of body fluid discharge, such as for use in the treatment of a condition responsive to increase of levels of antisecretory factor protein and/or antisecretory protein fragments in the blood of a patient, wherein said condition can be selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

The present disclosure further provides a method for treating ameliorating and/or preventing an abnormal physiological condition caused by pathologically high levels of body fluid discharge comprising administering to a subject and/or patient in need thereof a sufficient amount of a consumable product according to the present invention.

A method is herein disclosed for treatment, amelioration and/or prevention of a condition responsive to increased levels of antisecretory factor protein and/or antisecretory protein fragments in the blood of a patient comprising administering to a subject/patient in need thereof a sufficient amount of a consumable product according to the present invention, wherein said condition can be selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

In general, the consumable product disclosed herein may be provided as a food, feed, food supplement, feed supplement and/or a nutraceutical. The food may be food for human consumption such as but not limited to a functional food. The feed may be feed for animal consumption such as feed for poultry and/or livestock animals. The consumable product may be provided as a dry or semi-dry food and/or feed substance, or as a liquid. In one embodiment, the food and/or feed is provided as an infusion. Further, the consumable product may be a pharmaceutical product such as a medicament.

### Definitions and Abbreviations

Proteins are biological macromolecules constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides, polypeptides, or oligopeptides. The terms “protein”, “polypeptide”, “oligopeptide” and “peptide” may be used interchangeably in the present context. Peptides can have very few amino acid residues, such as between 2-50 amino acid residues (aa).

The term “antisecretory” refers in the present context to inhibiting or decreasing secretion and/or fluid transfer. Hence, the term “antisecretory factor (AF) protein” refers to a class of proteins capable of inhibiting or decreasing or otherwise modulating fluid transfer as well as secretion in a body.

In the present context, the terms an “antisecretory factor protein”, “antisecretory factor (AF) protein”, “AF- protein”, AF, or a homologue, derivative or fragment thereof, may be used interchangeably with the term “antisecretory factors” or “antisecretory factor proteins” as defined in WO 97/08202, and refer to an antisecretory factor (AF) protein or a peptide or a homologue, derivative and/or fragment thereof having antisecretory and/or equivalent functional and/or analogue activity, or to a modification thereof not altering the function of the polypeptide. Hence, it is to be understood that an “antisecretory factor”, “antisecretory factor protein”, “antisecretory peptide”, “antisecretory fragment”, or an “antisecretory factor (AF) protein” in the present context, also can refer to a derivative, homologue or fragment thereof. These terms may all be used interchangeably in the context of the present disclosure. Furthermore, in the present context, the term “antisecretory factor” may be abbreviated “AF”. Antisecretory factor (AF) protein in the present context also refers to a protein with antisecretory properties as previously defined in WO 97/08202 and WO 00/38535. Antisecretory factors have also been disclosed e.g. in WO 05/030246.

The term “ASP” is in the present context used for “antisecretory protein” i.e. natural antisecretory factor (AF) protein.

In the present context “AF activity” is measured as elevation of AF-Units in the blood after consumption of the consumable product of the present invention by inducing more than 0.5, such as at least 0.6, 0.7, 0.8, 0.9, 1, 1.5 or 2 AF-Units/ml blood in a human or an animal. Increased AF activity is defined by its effect on the fluid secretion into the small intestine of laboratory rats having been challenged with cholera toxin (RTT-test/ligated loop assay). One ASP/AF-Unit (FIL-Unit) corresponds to a 50% reduction of the fluid flow in the rat's intestine compared to a control without ASP, i.e. corresponding approximately to 1.5 nM AF protein per liter plasma (1.5 nM/L).

AF activity can also be measured by the use of a kit, an assay and/or a method as described in WO 2015/181324 (Antisecretory Factor Complex Assay) for verifying effectiveness of a consumable product according to the present invention as compliance of human and/or animals to the same consumable product after consumption.

By “functional food product” is meant, in the present context, a food product having a salubrious function, i.e. having a beneficial effect on the health of man or an animal.

In the present context, the expression “pathologically high levels of body fluid discharge” means levels of body fluid discharge such as from intracellular fluid and/or extracellular fluid, the latter being selected from the group consisting of intravascular fluid, interstitial fluid, lymphatic fluid and transcellular fluid, that deviate from what is considered normal and/or healthy in a human and/or animal. Specifically, the levels of body fluid discharge may be such that it may be considered by a health care professional such as a nurse or a physician appropriate to treat the patient. In the present context, the term “pathological” is used to in general describe an abnormal anatomical or physiological condition. The term “disease pathology” in general encompasses the causes, processes and changes in body organs and tissues that occur with human illness. Many of the most common pathological diseases are causes of death and disability.

AF: antisecretory factor,

Full-length AF protein (as shown in SEQ ID NO: 1)

AF-6: a hexapeptide CHSKTR (as shown in SEQ ID NO: 2);

AF-16: a peptide composed of the amino acids VCHSKTRSNPENNVGL (as shown in SEQ ID NO: 3);

AF-8: a septa peptide VCHSKTR (as shown in SEQ ID NO: 4);

Octa peptide IVCHSKTR (as shown in SEQ ID NO: 5);

RTT: Method for measuring a standardized secretion response in rat small intestine, as published in SE 9000028-2 (publication number 466331) for measuring content of AF (ASP) in blood.

g: gram(s)

ml: millilitre(s)

μL: microliter(s)

min.: minute(s)

vol: volume

UPLC: Ultra Performance Liquid Chromatography

V: Volt(s)

GHz: GigaHertz

LC-qTOF: Liquid Chromatography-quadrupole Time of Flight Mass Spectrometry (High Resolution Mass Spectroscopy)

RP: Reverse Phase

MS: Mass Spectroscopy

rpm: revolutions per minute

ppm: part per million

obiwarp—Ordered Bijective Interpolated Warping

mzML=mz(mass to charge ratio)

### BRIEF DESCRIPTION OF THE DRAWINGS

FIG. 1a shows the chemical structure of avenanthramides A, B, C, D, G, O, P and Q.

FIG. 1b shows the chemical structure of avenanthramide C methyl ester.

FIG. 2 shows the chemical structure of guaiacol.

FIG. 3 shows the chemical structure of ferulic acid.

FIG. 4 shows the chemical structure of sinapic acid.

FIG. 5 shows the chemical structure of L-tryptophan.

FIG. 6 shows the chemical structure of DL-phenylalanine.

FIG. 7 shows the chemical structure of (Z) N-feryloloyl alanine.

FIG. 8: Sequence listing

FIG. 9a shows the amount of avenanthramide C for the oat samples S1-S6.

FIG. 9b shows the amount of avenanthramide G for the oat samples S1-S6.

FIG. 10 shows the amount of (Z)-N-Feruloyl-5-hydroxyanthranilic acid for the oat samples S1-S6.

FIG. 11 a shows the amount of ferulic acid for the oat samples S1-S6.

FIG. 11 b shows the amount of sinapic acid for the oat samples S1-S6.

FIG. 11c shows the amount of p- coumaric acid for the oat samples S1-S6.

FIG. 12a shows the amount of L-tryptophan for the oat samples S1-S6.

FIG. 12b shows the amount of DL-phenylalanine for the oat samples S1-S6.

FIG. 13a shows the amount of avenanthramide C methyl ester for the oat samples S1-S6.

FIG. 13b shows the amount of avenanthramide A for the oat samples S1-S6.

FIG. 13c shows the amount of avenanthramide 1p, i.e. avenanthramide D, for the oat samples S1-S6.

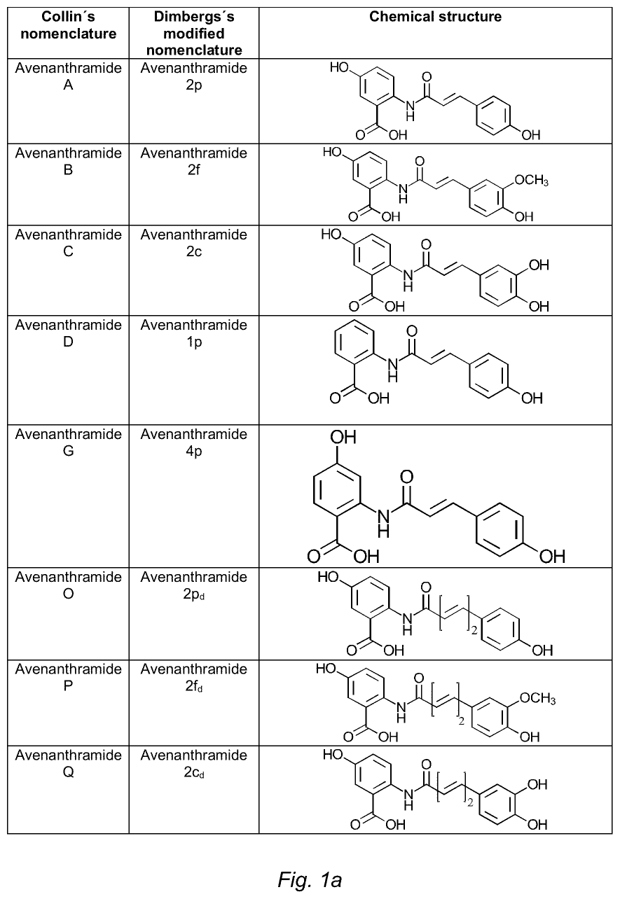

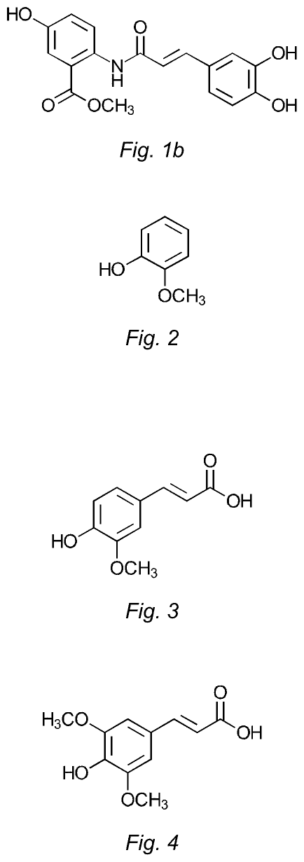

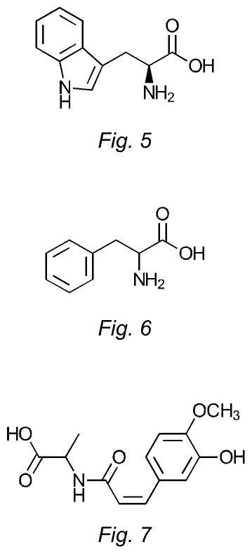

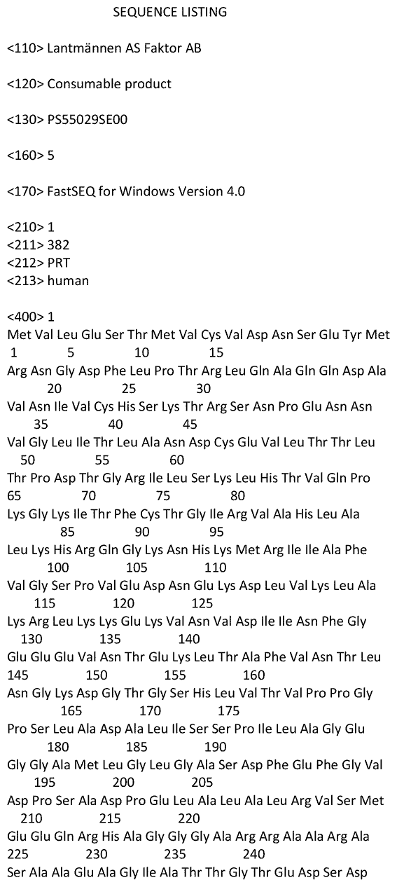

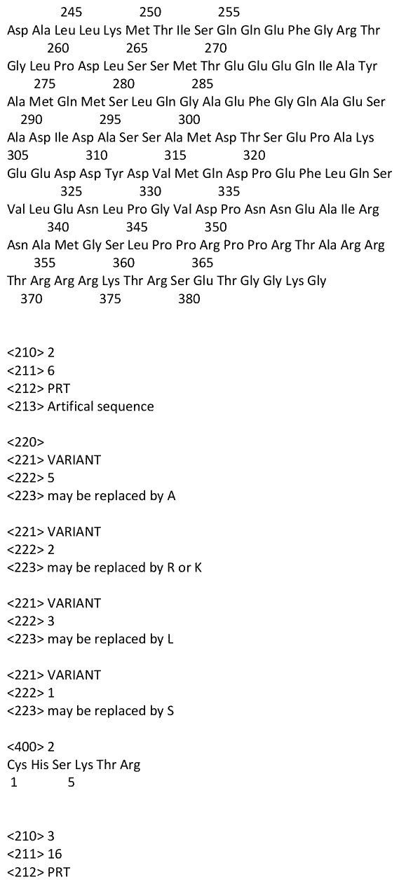

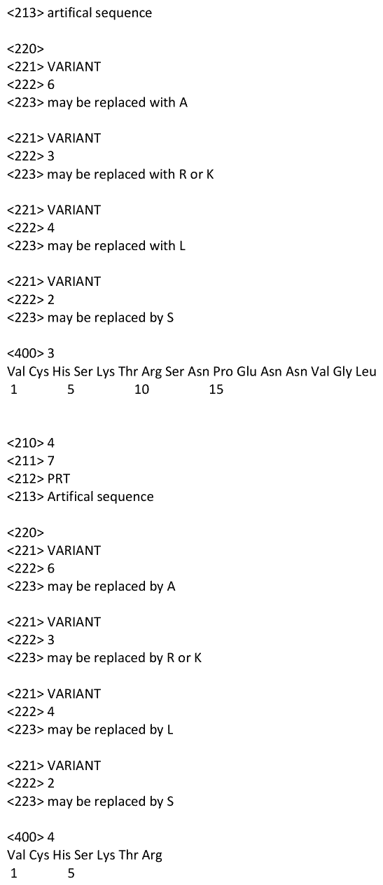

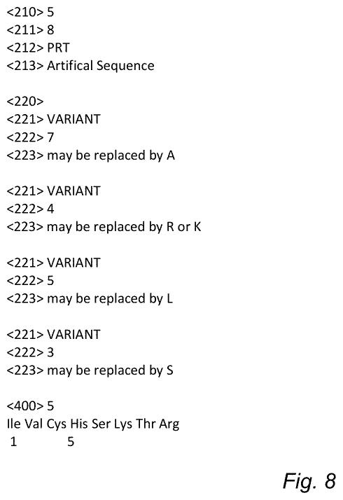

### DETAILED DESCRIPTION

Oats (Oats) is a well-known food or food ingredient. It is generally consumed as dehulled precooked (steamed) flakes or as oats flour. Oats is an important source for a number of valuable nutrients, among them β-glucans. β-glucans form very viscous water solutions, making worth filtering difficult. Oats also contains high levels of phytic acid, making essential mineral absorption in the gut less efficient.

The oats kernel is surrounded by a hard hull, considered as inedible. Consequently, a number of processes have been developed to dehull the oats kernel. Dehulling oats includes the risk of also removing the germ together with the hull. Hence oats intended for malting for e.g. beer brewing are not dehulled. Thus, as a rule, oats are malted with hull.

Seed development has produced an oats variety with an undeveloped hull, hull-less oat, also called “naked oat”. Naked oats is used, mainly as a food ingredient. Still, the lack of hull is necessarily compensated by the development of a strong pericarp.

The malting of oats has been investigated extensively, mainly with the purpose of improving worth yield and reducing of phytic acid content. During the malting process a vast number of dormant enzymes are activated, such as hydrolases, amylases, proteases, lipases and phytases.

Industrial malting consists of cleaning of the grain, steeping, germination, drying and sprout removal. The processes are performed batch-wise in grain beds. Moisture content of the grain in steeping is determined by contact time in water. Germination time is determined by the intended use of the finished malt, moisture content and temperature during germination. The generated metabolic heat is controlled by cooling with air. During germination, the grains are stirred by mechanical devices. Drying with warm or hot air induces the formation of taste and aroma substances.

Malting of seeds implies that the seeds are steeped in water for different length of time and temperatures. After steeping, the seeds are germinated for different lengths of time and temperature. As seeds are not sterile, malting also implies the growth of fungi and bacteria during steeping and germination. If the malted product is intended for beer production, the worth cooking functions also acts as a pasteurization. Hence, the growth of microorganisms can be controlled to a large extent. The heat evolved during germination is normally cooled by cold air blown through the grains.

When utilizing malted oats for other intended uses than in beer-production, though, the oats hulls make the product less palatable. What is more, malting of oats with hulls without pasteurisation can give a final product with unhealthy or less advantageous levels of microorganisms. Also, when cooking the worth, the hulls form a porous filter cake when the worth is filtered prior to fermentation.

Dehulling oats prior to malting would consequently reduce the problems listed above. But, dehulling dramatically increases the risk of removing the germ, making germination impossible. Further, moistened dehulled oats form impermeable beds due to the high level of hydrocolloids on the kernel surface.

To solve the problems referred to above a novel malting process is herein disclosed wherein a malted dehulled oats product is produced which is suitable for food, feed and/or medical food purposes. The malting process is described in detail in example 1.

The novel malting process described herein is a low-temperature malting process that allows malting of dehulled oats in a process that is easily scalable to industrial use.

In the process, the oats lot is refined by sieving and by using gravity tables so that the final 1000 grain weight exceeds 30 grams/1000 kernels. Such as that the final 1000 grain weight exceeds 30, 31, 32, 33, 34, 35, 36, 37, 38, 39, 40, 41, 42, 43, 44, 45, 46, 47, 48, 49, or 50 grams/1000 kernels.

The selected oats lot is dehulled by a dehuller. In the disclosed process, the dehuller is preferably a rotating disc with radial groves, but the person skilled in the art will understand that any commercially available dehuller can be used, as long as it leaves dehulled oats with the specified minimum germinability. A commercially available dehuller can be selected from the non-limiting group of Bailer BSSA Stratopact HKE5OHP Ex and Streckel &Schrader. The feed and disc speed are typically selected so that 30 -70% of the kernels are dehulled at each passage.

The germinability of the dehulled oats is tested to exceed 95%, such as no less than 80.

81, 82, 83, 84, 85, 85, 87, 88, 89, 90, 91, 92, 93, 94 or 95% in petri-dish, or at least 82%, such as at least 77, 76, 78, 79, 80, 81 or 82% in H₂O₂.

The selected dehulled oats kernels are steeped with cold water (w), optionally alternatingly in dried conditions (d) at temperatures between 5-15° C., or 7° C.-15° C., such as at temperatures not exceeding 5, 6, 7, 8, 9, 10, 11, 12, 13, 14 or 15° C., such as at a temperature between 5-12° C., 5-15, 12° C., 7-12° C., 12-15° C., 10-15° C. or 7-10° C., fora total of 1-3 days, such as for 20-26 hours, such as for 20, 21, 22, 23, 24, 25 or 26 hours, such as for no less than 1, 2 or 3 days. Kernel moisture content is herein kept between 30-50%, such as between 30-35%, 30-40%, 30-45%, 35-40%, 35-45%, 35-50%, 40-45%, 40-50% or 45-50%. The kernel moisture should in this process step not exceed 30, 35, 40, 45 or 50%.

In the present context, the malting comprises wet steeping in which the oats is partly or entirely soaked with water. Additionally, or alternatively, the wet steeping may involve spraying with water.

After steeping, the dehulled oats is germinated for 7-9 days at 5-20° C., preferably at 7-12° C., at 7-15° C., or at 12-15° C., such as for at least 7, 8 or 9 days at a temperature not exceeding 12, 13, 14, 15 or 20° C., such as at a temperature of 5, 6, 7, 8, 9, 10, 11, 12, 13, 14,15 or 20° C.

The heat evolved is cooled by cold air. Due to the impermeable beds that can be formed, only shallow beds are used, with no more than 0.5 m bed height, such as with max 0.1, 0.2, 0.3, 0.4 or 0.5 m bed height. Any movement of the grains is performed at slow speed.

The germinated grain is initially dried at low air temperature not exceeding 35° C., such as at a temperature between 15-35, 20-35, 25-35 or 30-35° C. In the later stages of drying, when moisture content is below 20%, drying air temperature is raised to a maximum temperature of 65° C., max 65-70° C. or max 65-80° C. The drying air temperature should not exceed 80° C. at any time.

By this novel malting method, a healthy malted dehulled oats product with a high level of enzymatic activity is produced as disclosed in the present disclosure and as analysed in example 2 herein.

It has been found that the process for malting the oats impacts the properties of the consumable product into which it is incorporated. Importantly, the malting should take place at a low temperature such as from about 5° C. to about 20° C. and subsequent drying should take place at an air temperature of 80° C. or less. It will be appreciated that in this document the expression “a temperature of 80° C. or less” means a temperature equal to or less than 80° C.

Thus, there is provided a consumable product as described herein, wherein the malted dehulled oats is obtained from a process comprising the steps of:
a. malting dehulled oats at a temperature from about 5° C. to about 20° C., and
b. drying said dehulled oats at no more than 80° C.

In a further example, there is provided a consumable product as described herein, wherein the malted dehulled oats is obtained from a process comprising the steps of:
a. wet steeping of dehulled oats at a temperature from about 5° C. to about 20° C.,
b. germinating/growing at a temperature from about 5° C. to about 20° C.,
c. optionally repeating any one of steps a-b, and subsequent
d. drying of said dehulled oats at no more than 80° C.

Steps a. and/or b. described herein may independently take place at a temperature of about 8° C. or from about 13° C. to about 15° C.

The present disclosure is based on the unexpected and surprising finding that a consumable product comprising malted dehulled oats, produced with a malting process according to the present invention, comprises a combination of (i) avenanthramide A, (ii) avenanthramide C methyl ester, (iii) avenanthramide D and (iv) certain compounds as described herein to such an increased amount that it induces endogenous production of antisecretory factor (AF) protein and/or fragments thereof in a subject after consumption.

Surprisingly, it was found that that the combination of the compounds (i)-(iv) in the concentrations described herein increases the Antisecretory Factor (AF) activity, and/or improves the endogenous formation of AF in a subject after consumption.

Thus, there is provided a consumable product comprising malted dehulled oats and/or a leachate of said malted dehulled oats comprising in particular (i) avenanthramide D, wherein the concentration of (i) is higher as compared to the corresponding non-malted dehulled oats, and wherein the consumable product induces endogenous production of antisecretory factor (AF) protein and/or fragments thereof in a subject after consumption.

The malted dehulled oats and/or a leachate of said malted dehulled oats comprised in the consumable product may further comprise one or more of:

(ii) avenanthramide A,

(iii) avenanthramide C,

(iv) avenanthramide C methyl ester,

(v) (Z)-N-feruloyl 5-hydroxyanthranilic acid, and optionally

(vi) avenanthramide G;

wherein the concentration of one or more of (ii), (iii), (iv) (v) and (vi) is higher as compared to the corresponding non-malted dehulled oats.

The malted dehulled oats and/or a leachate of said malted dehulled oats comprised in the consumable product may further comprise:

(vii) a compound selected from the group consisting of guaiacol or a derivative thereof, L-tryptophan, DL-phenylalanine, and any combination thereof;

wherein the concentration of one or more of (vii) is higher as compared to the corresponding non-malted dehulled oats.

The guaiacol derivative described herein may be ferulic acid, sinapic acid and/or p-coumaric acid.

The consumable product described herein may comprise malted dehulled oats and/or a leachate thereof in an amount sufficient to induce endogenous production of antisecretory factor (AF) protein and/or fragments thereof in a subject after consumption. The specific amount of the consumable product may be adjusted depending on the condition to be treated. For instance, the consumable product may comprise malted dehulled oats and/or a leachate thereof in an amount sufficient to increase the amount of antisecretory protein and/or fragments thereof in the subject's blood to more than 0.5 Units/ml blood, such as to at least 0.6, 0.7, 0.8, 0.9 or at least 1 Units/ml blood. The skilled person may determine the amount using methods known in the art such as the RTT method and/or the Antisecretory Factor Complex Assay described herein.

The consumable product described herein may be food, feed, a food supplement, and/or a nutraceutical. The food or feed may be for human and/or animal consumption. Generally, food is intended for human consumption while feed is intended for animal consumption. The consumable product described herein may be a liquid, a solid and/or a combination thereof. For instance, the liquid may be a beverage. In a further example, the consumable product may be an infusion. When the food or feed is a solid it may be dry or semi-dry.

The food described herein may be a medical food. Additionally, or alternatively, the food described herein may be a FSMP, i.e. a food for special medical purposes. It will be appreciated that a FSMP may be food for individuals who suffer from certain diseases, disorders and/or medical conditions, and/or for people whose nutritional requirements cannot be met by normal foods. In a further example, the food described herein may be a nutraceutical. As used herein, a nutraceutical is a food or feed providing an extra health benefit in addition to basic nutritional value in food or feed. The food and/or food supplement for human consumption may be in the form of a liquid, a solid or a combination thereof. In an example, the food for human consumption may be in the form of a liquid, i.e. a liquid food for humans

The feed described herein may be given to animals such as poultry or livestock animals. The feed for animals may be in the form of a liquid, a solid or a combination thereof. In an example, the feed for animals may be in the form of a liquid, i.e. a liquid feed for animals. Examples of poultry include chickens, hens, ducks, geese, pigeons, quails, turkeys, pheasants and ostriches. Examples of livestock animals include cattle such as cows, horses, donkeys, goats, pigs and sheep. In a further example, animals that can be treated with the consumable product described herein include camels, deer, elks, yaks, lamas, alpacas and water buffalos. In still a further example of animals that can be treated with the consumable product described herein include pets such as dogs, cats, rabbits, guinea pigs and hamsters. In a particular example, the feed described herein is horse feed. In a further example, the feed described herein is pig feed. In still a further example, the feed described herein is dog or and/or cat feed. In still a further example, the feed described herein is fish feed.

Moreover, it will be appreciated that the consumable product described herein may be feed for ruminants such as cows, sheep and/or camels. The feed for ruminants may be in the form of a liquid, a solid or a combination thereof. In an example, the feed for ruminants may be in the form of a liquid, i.e. a liquid feed for ruminants.

In the present context, the term “feed” is used to describe materials of nutritional value fed to animals. Each species has a normal diet composed of feeds or feedstuffs which are appropriate to its kind of alimentary tract and which are economically sensible as well as being nutritious and palatable. Animals such as agricultural animals at pasture often have a diet which is very variable and subject to naturally occurring nutritional deficiencies. The feed disclosed herein may help to remedy or at least alleviate such deficiencies as well as disease, condition and/or symptom brought on by a stressful situation and or environment.

The presently disclosed feed can further comprise forage feed, such as hay, ensilage, green chop. i.e. any feed with a high cellulose content relative to other nutrients. The presently disclosed feed can further comprise feed grain such as cereal and other grains and pulses used as animal feed. The aforementioned feed grain may include wheat, barley, oats, rye, maize, peas, raps, rape seed, rape seed meal, soybean meal, and sorghum.

In a further example, the feed described herein may be provided in pelleted form.

The presently disclosed feed can further comprise feed supplements, i.e. nutritive materials which are feedstuffs in their own right, and which are added to a basic diet such as pasture and/or forage to supplement its deficiencies, such as minerals and aromatics. Feed supplements typically include trace elements and macrofeeds, feed additives or supplements, such as protein supplements and/or minor feed ingredients, such as essential amino acids and vitamins.

The consumable product can be a feed supplement in itself.

Albeit the present disclosure mainly is directed to a consumable product in the form of food or feed, it is also envisaged that the consumable product may be administrated to a subject in other ways than oral intake. For instance, the consumable product may be provided in a form making it suitable for topical, ocular, subcutaneous and/or systemic administration.

The food described herein may form part of a functional food. For instance, the functional food may be muesli, bread, biscuits, gruel, oatmeal, grains, flakes, pasta, omelette and/or pancake. In an example, the functional food is a beverage, or a food intended to drink. Alternatively, the functional food is not a beverage, or a food intended to drink but a solid or semi-solid foodstuff

Due to the presence of the malted dehulled oats and/or leachate of malted dehulled oats as described herein, the consumable product such as the food and/or feed possesses properties associated with induction of antisecretory factor (AF) protein and/or fragments thereof such as anti-diarrhoeal properties and/or anti-inflammatory properties.

Consequently, the consumable product may be used in treatment, prevention and/or prophylaxis of abnormal physiological conditions caused by pathologically high levels of body fluid discharge. Additionally, or alternatively, the consumable product may be used in the treatment, prevention and/or prophylaxis of a condition which is responsive to increase of antisecretory factor protein and/or antisecretory protein fragments in the blood of a patient. The condition(s) described herein may be selected from the group consisting of diarrhoea, inflammatory diseases, oedemas, autoimmune diseases, cancer, tumours, leukaemia, diabetes, diabetes mellitus, glioblastoma, traumatic brain injury, intraocular hypertension, glaucoma, lipid raft dysfunction, compartment syndrome, Alzheimer's disease, Parkinson's disease, encephalitis, and Meniere's disease.

The consumable product described herein may be provided in the form of a medicament. Thus, there is provided a consumable product as described herein such as a functional food product and/or a pharmaceutical product for use as a medicament.

The present disclosure will be further explained hereinafter by means of non-limiting examples and with reference to the appended drawings.

### REFERENCES

1. Lange S. and Lönnroth I., International Review of Cytology, Vol 210 (2001), 39-75
2. WO 97/08202;
3. WO 05/030246;
4. WO 2007/126364;
5. WO 2018/015379
6. WO 98/21978
7. WO 07/126363
8. SE 9000028-2 (publication No. 466,331)
9. A study of avenanthramides in oats for future applications” by Eléne Karlberg, Uppsala University School of Engineering, published in June 2010
10. WO 2010/108277
11. WO 2015/179676
12. WO 2007/52153
13. US 4,581,847
14. WO 2007/117815
15. WO 2017/09004
16. WO 00/38535
17. WO 201 5/1 81 324
18. Food Chemistry 253 (2018) 93-100, section 2.5 page 95
19. Chambers, M. et al. (2012) A cross-platform toolkit for mass spectrometry and proteomics. Nat. Biotechnol., 30, 918-920.
20. Smith, C. A. et al. (2006) XCMS: processing mass spectrometry data for metabolite profiling using nonlinear peak alignment, matching, and identification. Anal. Chem., 78, 779-787.
21. Stanstrup, J. et al. (2013) Metabolite profiling and beyond: approaches for the rapid processing and annotation of human blood serum mass spectrometry data. Anal. Bioanal. Chem., 405, 5037-5048.
22. Zhu, Z.-J. et al. (2013) Liquid chromatography quadrupole time-of-flight mass spectrometry characterization of metabolites guided by the METLIN database. Nat. Protoc., 8, 451-460.
23. Ganna, A. et al. (2016) Large-scale non-targeted metabolomic profiling in three human population-based studies. Metabolomics, 12, 4.
24. Lin Shi, Johan A Westerhuis, Johan Rosén, Rikard Landberg, C. and Brunius (2018) Variable selection and validation in multivariate modelling. Bioinformatics.
25. Shi, L. et al. (2018) Plasma metabolites associated with type 2 diabetes in a Swedish population: a case—control study nested in a prospective cohort. 849-861.
26. Brunius, C. et al. (2016) Large-scale untargeted LC-MS metabolomics data correction using between-batch feature alignment and cluster-based within-batch signal intensity drift correction. Metabolomics, 12, 173.
27. Stekhoven, D. J. and Bühlmann, P. (2012) Missforest-Non-parametric missing value imputation for mixed-type data. Bioinformatics, 28, 112-118.
28. De Bruijn, W. J. C. et al. (2016) Mass Spectrometric Characterization of Benzoxazinoid Glycosides from Rhizopus-Elicited Wheat (Triticum aestivum) Seedlings. J. Agric. Food Chem., 64, 6267-6276
29. Hanhineva, K. et al. (2011) Qualitative characterization of benzoxazinoid derivatives in whole grain rye and wheat by LC-MS metabolite profiling. J. Agric. Food Chem., 59, 921-927
30. Koistinen, V. M. et al. (2018) Metabolic profiling of sourdough fermented wheat and rye bread. Sci. Rep., 8, 1-11.
31. Sumner, L. W. et al. (2007) Proposed minimum reporting standards for chemical analysis. Metabolomics, 3, 211-221.

### EXAMPLES

### Example 1

### The Novel Oat Malting Process The aim of this experiment was to find a new low-temperature malting process that would allow malting of dehulled oats in a scale-able process.

The oats lot is refined by sieving and by using gravity tables so that the final 1000 grain weight exceeds 30 grams/1000 kernels.

The germinability is tested to exceed 95% in petri-dish, or at least 82% in H₂O₂.

The selected oats lot is dehulled by a dehuller (Bühler BSSA Stratopact HKE50HP Ex). The feed and disc speed are selected so that 30 -70% of the kernels are dehulled at each passage.

Dehulled kernels with germs are sorted out by gravity tables. The germinability of the dehulled kernels is tested to exceed 95%, or at least 82% in H₂O₂.

The selected dehulled oats kernels are steeped with cold water (w) at temperatures between 7° C. and 15° C. and in dry conditions (d), for a total of 1-3 days (20-26hours) (2w+10d+2w+10d+2w=26/20h). Kernel moisture content is between 30-50%.

After steeping, the dehulled oats is germinated for 7-9 days at 12-15° C. The heat evolved is cooled by cold air. Due to the impermeable beds that can be formed, only shallow beds are used, with max 0.5 m bed height. Any movement of the grains is performed at slow speed.

The germinated grain is initially dried at low air temperature, max 35° C. In the later stages of drying, when moisture content is below 20%, drying air temperature is raised to max. 65° C.

By this novel malting method, a healthy malted dehulled oats product with a high level of enzymatic activity is produced.

TABLE 1
Micromalting

| | JW 281 | | | | |
|---|---|---|---|---|---|
| MICROMALTING | Kaura | | 6.3.-17.3.2004 | | |
| Box number | 1 | 2 | 3 | 4 | 5 |
| OATS ANALYSIS | | | | | |
| Moisture % | 12.2 | 12.2 | 12.2 | 12.2 | 12.2 |
| Protein % | — | — | — | — | — |
| Germinat.capacity | 82 | 82 | 82 | 82 | 82 |
| (H₂O₂) % | | | | | |
| Sorting mm | 1.5 | 1.5 | 1.5 | 1.5 | 1.5 |
| Fraction I, > 2.8 | — | — | — | — | — |
| mm % | | | | | |
| Fraction II, > 2.5 | — | — | — | — | — |
| mm % | | | | | |
| MALTING PROCESS | | | | | |
| Steeping program | 2 w + 10 d + 2 w + 10 d + 2 w = 26/20 h | | | | |
| Wet/dry steeping | 15/15 | | | | |
| temp. ° C. | | | | | |
| Moisture after 1. wet | — | 30.5 | 30.5 | 30.6 | 30.5 |
| steep % | | | | | |
| Moisture after 2. wet | — | — | — | — | — |
| steep % | | | | | |
| Moisture after | 42.0 | 42.9 | 43.1 | 43.4 | 43.2 |
| steeping % | | | | | |
| Spraying day | 1 | 1 | 1 | 1 | 1 |
| Moisture after | 46 | 46 | 46 | 50 | 46 |
| spraying % | | | | | |
| Germination program | 9/12 | 7/15 | 7/15 | 7/15 | 7/15 |
| days/° C. | | | | | |
| Germination time | 9 | 7 | 7 | 7 | 7 |
| days | | | | | |
| Germination 2 days/% | 94/79 | 70/79 | 76/80 | 74/71 | 80/74 |
| Green malt | 32.4 | 42.2 | 42.5 | 46.6 | 42.6 |
| moisture % | | | | | |
| Kilning program | EM* | PM | EM | EM | freeze |
| | | | | | drying |
| Respiration | 5.0 | 5.3 | 7.2 | 9.0 | 7.7 |
| losses % | | | | | |
| Rootlet losses % | 2.1 | 5.3 | 5.2 | 5.0 | 11.2 |
| Total losses % | 7.1 | 10.6 | 12.4 | 14.0 | 18.9 |

*EM (Enzyme malting)

### Example 2

In this example, analysis was performed on 6 oat samples. Sample 51 was un-malted oat, i.e., oats that had not been subjected to malting, with hull. Sample S2 was oats with hull that had been subjected to malting. Sample S3 was dehulled oats that had been subjected to malting. Sample S4 was un-malted dehulled oat. Sample S5 was naked oats that had been subjected to English malting. Sample S6 was dehulled oats that had been subjected to Nordic malting, i.e., a novel malting process as described in this document.

Oat sample extracts were thawed at room temperature for 30 min and a 100 μL aliquot of each sample was transferred into a 1.5 ml microcentrifuge tube. Cold extraction solution (900 μL) was mixed with samples using a multi-tube vortexer (VWR International, Inc) for 10 min and incubated at 4° C. for 2 h. The mixtures were centrifuged for 12 min at 13000 rpm at 4° C. The supernatant from each sample was kept in refrigerator at 4° C. until they were injected on the LC-MS instrument. Each oat sample was prepared in triplicates. Quality control samples (QC) were achieved by pooling aliquots of all the study oat samples (i.e., 6 varieties with and without treatments) and were used to monitor the stability and functionality of the system throughout the instrumental analyses.

### Analytical Protocol of Untargeted LC-MS Metabolomics

Oat extract samples were analyzed by LC-qTOF mass spectrometry -MS (Agilent Technologies 6550 iFunnel Q-TOF LC/MS, United States). Sample solution (5 μL) was injected for reversed-phase (RP) chromatographic analyses using both positive and negative electrospray ionization modes. Separation was performed using an Acquity UPLC High Strength Silica T3 column (2.1×100 mm, 1.8 μm; Waters) at 45° C. The mobile phase was delivered at 400 μL/min and consisted of eluent A (water, Milli-Q purified; Millipore) and eluent B (methanol, Sigma-Aldrich), both containing 0.04% (vol:vol) of formic acid (Sigma-Aldrich), delivered in a profile: 0-10.5 min 100% B, 10.5-15 min: 5% B. The dual electrospray ionization source (ESI) was operated using the following conditions: Drying gas (nitrogen) temperature of 175° C. and flow of 10 L/min, nebulizer pressure of 45 PSI, capillary voltage of 3500 V, fragment or voltage of 175 V, and a skimmer of 65V. For data acquisition, a 2-GHz extended dynamic range mode was used, and the instrument was set to acquire over the mass range of m/z 50-1700. Data were collected in centroid mode at an acquisition rate of 1.67 spectra/s with an abundance threshold of 200 counts. The automatic data-dependent MS/MS analyses were performed on the QC samples, and the 4 most abundant ions were selected for fragmentation from every precursor scan cycle.

Collision energies were 10, 20 and 40 volt (V). Continuous mass axis calibration was performed by monitoring two reference ions, m/z 121.050873 and m/z 922.009798 for positive mode and m/z 112.98558700 and 966.000725 for negative mode, from an infusion solution throughout the runs. All the oat samples were analysed randomly in one batch. Two blank samples and one priming quality control sample provided by the Chalmers Mass Spectrometry Infrastructure were injected before the analytical sequence. Two pooled QCs described as above were injected at the beginning and end and as every 10^{th} injection throughout the sequence.

### Detection and Quantification of Avenanthramides

The method workup was identical, but the mass spectrometer used for analysis differed. The detection and quantification were performed as described in Food Chemistry 253 (2018) 93-100 section 2.5 page 95. The LC-MS/MS system used was a QTRAP 6500+ LC-MS/MS (SCIEX A/B, Stockholm, Sweden). Avenanthramides were ionized using positive electrospray ionization in multiple reaction monitoring (MRM) mode for each of the avenanthramides, which were as follows: B (2c) m/z 329.9→176.9 (collision energy (CE)-15 V); C (2f): m/z 315.9→162.9 (CE-15 V); A (2p): m/z 299.9→146.9 (CE-25 V); 2fd: m/z 342→172.95 (CE-10 V) and 2pd m/z 326→173 (CE-12 V). Dwell times were 50 ms. For all mass spectrometry analyses, the ion source temperature was set to 500° C., entrance potential 10 V and drying curtain gas flow 30 L/min. Identity of avenanthramides was confirmed using neutral loss scanning for loss of m/z 153, which is characteristic of the main avenanthramides (Xie et al., 2017).

### Data Pre-Processing

Raw data files from RP (ESI+), RP (ESI−) were converted to mzML format using ProteoWizard msconvert (Chambers et al., 2012). Data deconvolution was performed with xcms, a freely available software under open-source license, implemented in R (Smith et al., 2006). Specifically, feature detection in each chromatogram was performed using the centWave algorithm implemented in the xcmsSet function and obiwarp was applied for retention time correction. The term ‘feature’ refers to a mass spectral peak, i.e. a molecular entity with a unique mass-to-charge ratio and retention time as measured by an LC-MS instrument. Parameters were the values suggested by xcms online (https://xcmsonline.scripps.edu/) and from recently relevant publications (Stanstrup et al., 2013; Zhu et al., 2013; Ganna et al., 2016; Shi et al., 2018). Parameters were: peak width=c(10, 60), ppm=15, prefilter intensity (3, 1000), bandwidth (2), mzdiff (0.01). Quality of data acquisition and processing was examined by visualization of the total ion chromatogram and the base peak chromatogram for each sample, extracted-ion chromatograms for multiple features, and assessment of differences between adjusted and raw retention times per sample. Within-batch signal intensity normalization was performed using R package ‘batchcorr’ (Brunius et al., 2016). Features passing a QC test (CV<0.3) were determined as qualified features and were further subjected to statistical analyses. In total, 3511 and 3809 features were retained after a stringent normalization procedure for RP (ESI+) and RP (ESI−), respectively. Missing values were imputed by using random forest algorithm implemented in R package ‘missForest’ (Stekhoven and Bühlmann, 2012).

### Metabolite Identification

Metabolite identification was accomplished based on accurate mass and MS/MS fragmentation matched against online databases (i.e. Metlin, FooDB and MassBank) or the literature (De Bruijn et al., 2016; Hanhineva et al., 2011; Koistinen et al., 2018). The confidence level of annotation was categorized according to the Metabolomics Standard Initiative (MSI) (Sumner et al., 2007).

### Results

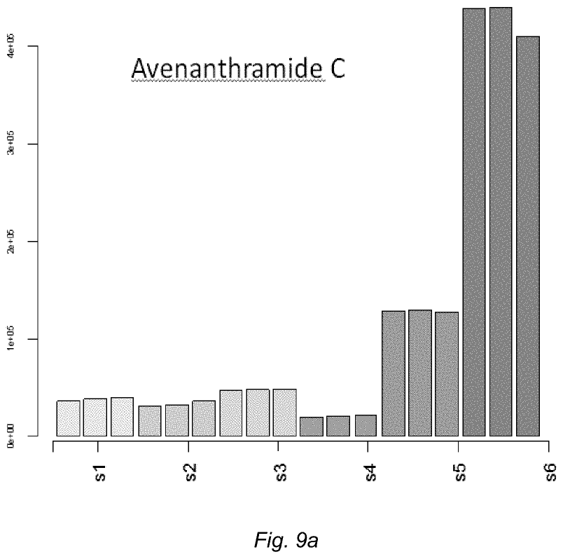

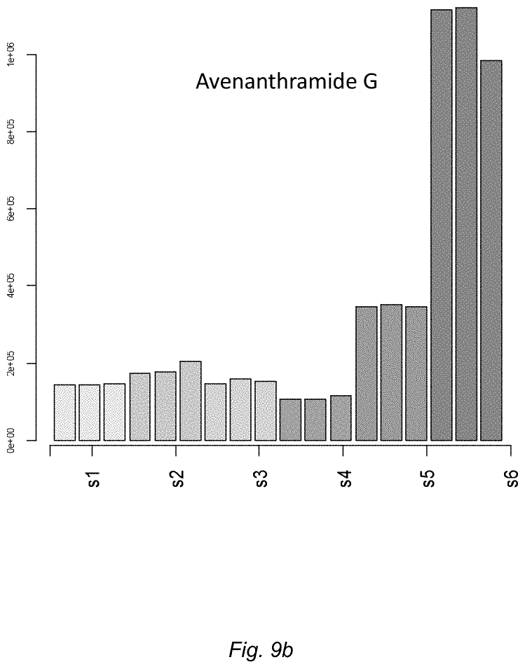

FIG. 9a shows the amount of avenanthramide C for the oat samples S1-S6. The amount of avenanthramide C was found to increase significantly for S5 and S6. In particular, the Nordic malting increased the amount of avenanthramide C as shown for S6.

FIG. 9b shows the amount of avenanthramide G for the oat samples S1-S6. The amount of avenanthramide G was found to increase significantly for S5 and S6. In particular, the Nordic malting increased the amount of avenanthramide G as shown for S6.

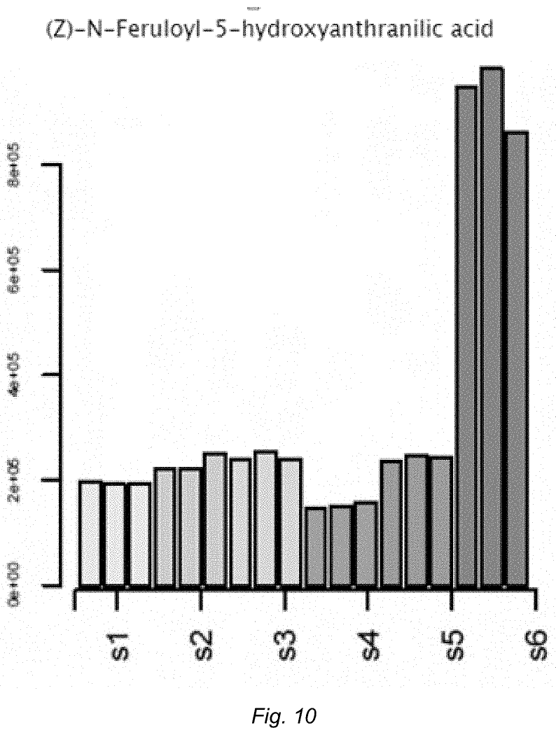

FIG. 10 shows the amount of (Z)-N-Feruloyl-5-hydroxyanthranilic acid for the oat samples S1-S6. In particular, the Nordic malting increased the amount of (Z)-N-Feruloyl-5-hydroxyanthranilic acid as shown for S6.

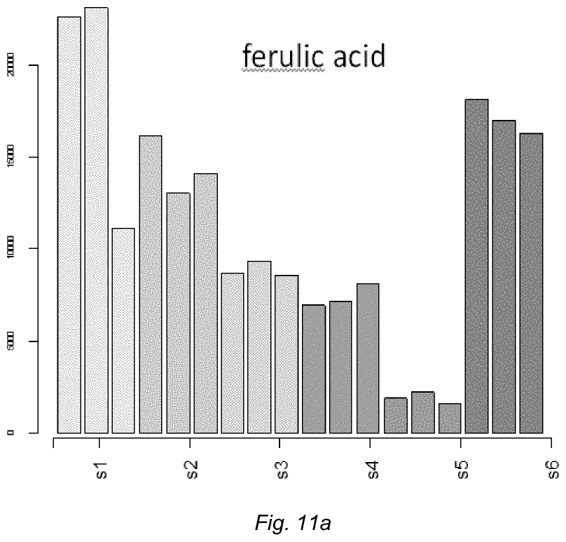

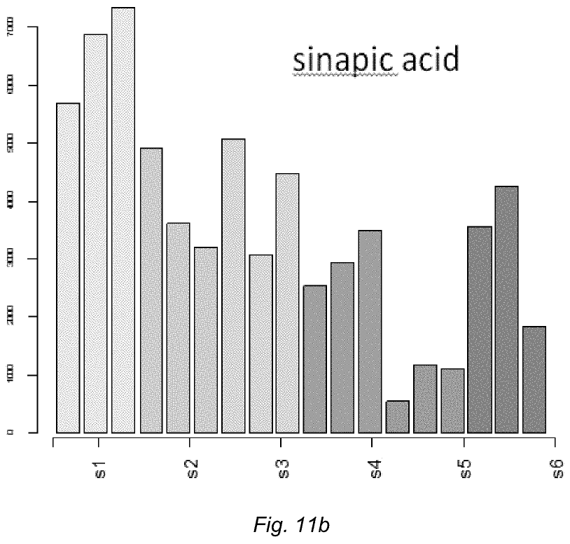

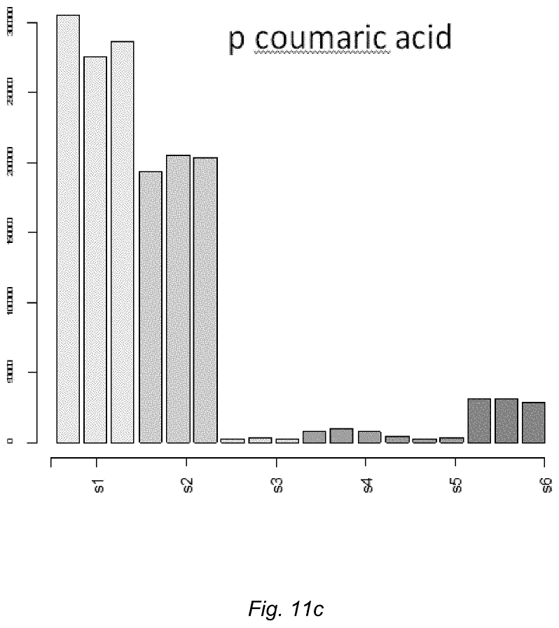

FIG. 11a shows the amount of ferulic acid for the oat samples S1-S6. It was observed that the Nordic malting (S6) increased the amount of ferulic acid more than the English malting (S5).

FIG. 11b shows the amount of sinapic acid for the oat samples S1-S6. It was observed that the Nordic malting (S6) increased the amount of sinapic acid more than the English malting (S5).

FIG. 11c shows the amount of p-coumaric acid for the oat samples S1-S6. It was observed that the Nordic malting (S6) increased the amount of p-coumaric acid more than the English malting (S5).

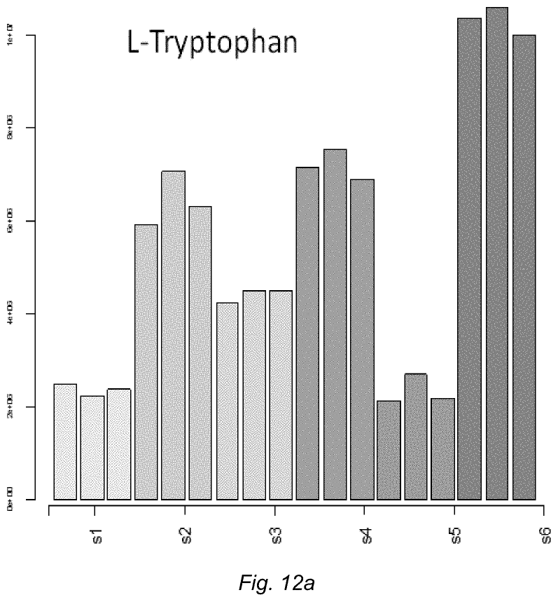

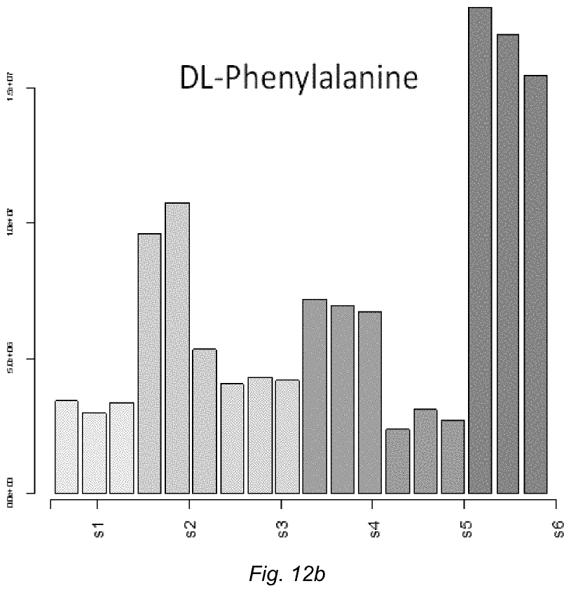

FIG. 12a shows the amount of L-tryptophan for the oat samples S1-S6. It was observed that the Nordic malting (S6) increased the amount of L-tryptophan more than the English malting (S5).

FIG. 12b shows the amount of DL-phenylalanine for the oat samples S1-S6. It was observed that the Nordic malting (S6) increased the amount of DL-phenylalanine more than the English malting (S5).

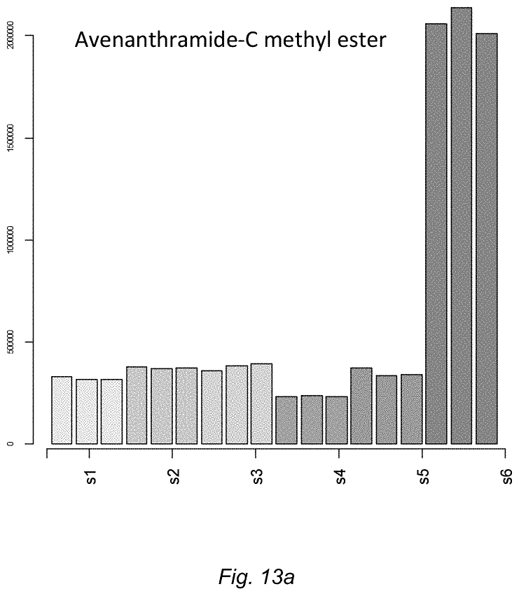

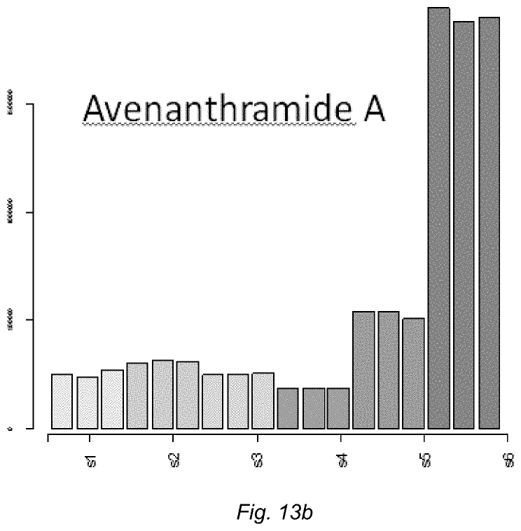

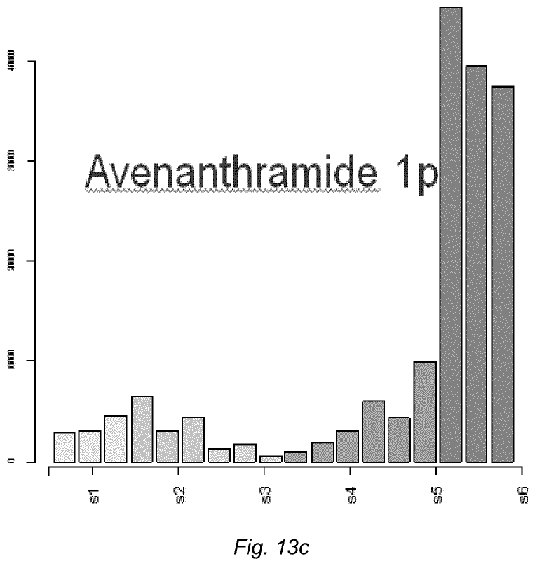

FIG. 13a shows the amount of avenanthramide C methyl ester for the oat samples S1-S6. It was observed that the Nordic malting (S6) increased the amount of avenanthramid C methyl ester more than the English malting sample S5, and also more than samples S1-S4.

FIG. 13b shows the amount of avenanthramide A for the oat samples S1-S6. It was observed that the Nordic malting (S6) increased the amount of avenanthramide A more than the English malting sample S5, and also more than samples S1-S4.

FIG. 13c shows the amount of avenanthramide 1p, i.e., avenathramide D, for the oat samples S1-S6. It was observed that the Nordic malting (S6) increased the amount of avenanthramide 1p, i.e. avenanthramide D, more than the English malting sample S5, and also more than samples S1-S4.

It will be appreciated that they axis in FIGS. 9-13 shows the detector response of the metabolites.

What is claimed is:

1. A consumable product comprising malted dehulled oats and/or a leachate of said malted dehulled oats, wherein said malted dehulled oats are produced by a malting process characterized by comprising the steps of:
a. dehulling oat kernels,
b. wet steeping of the dehulled oat kernels at a temperature from 5° C. to 20° C.,
c. germinating of said dehulled oat kernels at a temperature from 5° C. to 20° C.,
d. optionally repeating any one of steps b-c, and subsequent
e. drying of said dehulled oat kernels at no more than 80° C. air temperature,
wherein the malted dehulled oats comprise avenanthramide D at a higher concentration as compared to the corresponding non-malted dehulled oats and wherein the consumable product induces endogenous production of antisecretory factor (AF) protein and/or fragments thereof in a subject after consumption.

2. A consumable product according to claim 1, wherein the wet steeping of the dehulled oat kernels in step b. is performed at a temperature from 7° C. to 15° C. for 1-5 days.

3. A consumable product according to claim 1, wherein the germinating of said dehulled oat kernels in step c. is performed for 5-9 days at a temperature of 12-15° C.

4. A consumable product according to claim 1, wherein the germinating of said dehulled oat kernels in step c. is performed for 7 days at a temperature not exceeding 15° C.

5. A consumable product according to claim 1, wherein the malted dehulled oats comprise:
(i) avenanthramide D,
wherein the concentration of (i) is at least 100% higher as compared to non-malted dehulled oats.

6. A consumable product according to claim 1, wherein the malted dehulled oats further comprise one or more of:
(ii) avenanthramide A,
(iii) avenathramide C,
(iv) avenanthramide C methyl ester,
(v) (Z)-N-feruloyl 5-hydroxyanthranilic acid, and
(vi) avenanthramide G, and
wherein the concentration of one or more of (ii), (iii), (iv), (v) and (vi) is higher as compared to non-malted dehulled oats.

7. A consumable product according to claim 1, wherein the malted dehulled oats further comprises:
(vii) a compound selected from the group consisting of guaiacol or a derivative thereof, L-tryptophan, DL-phenylalanine, and any combination thereof, wherein the concentration of one or more of (vii) is higher as compared to in the corresponding non-malted dehulled oats.

8. A consumable product according to claim 7, wherein the guaiacol derivative is ferulic acid, sinapic acid, or p-coumaric acid.

9. A consumable product according to claim 1, wherein said consumable product comprises malted dehulled oats and/or a leachate of said malted dehulled oats in an amount sufficient to increase the amount of antisecretory protein and/or fragments thereof in the subject's blood to at least about 1 unit/mL, and/or to increase the amount of ASP Units in the subject's blood to at least about 1 Unit/ml.

10. A consumable product according to claim 1, which is a food, feed, a food supplement and/or a nutraceutical.

11. A consumable product according to claim 1, which is in the form of a liquid, a solid or a combination thereof.

12. A consumable product according to claim 1, which has antisecretory properties, anti-diarrhoeal properties and/or anti-inflammatory properties.

13. A method for treatment, amelioration and/or prevention of a condition responsive to increase of levels of antisecretory factor protein and/or antisecretory protein fragments in the blood of a patient comprising administering to a subject/patient in need thereof a sufficient amount of a consumable product according to claim 1.

14. A method for treatment, amelioration and/or prevention of a condition according to claim 13, wherein said condition is selected from the group consisting of diarrhoea, inflammatory disease, oedema, autoimmune disease, cancer, tumour, leukaemia, diabetes, diabetes mellitus, glioblastoma, traumatic brain injury, intraocular hypertension, glaucoma, compartment syndrome, Alzheimer's disease, Parkinson's disease, encephalitis, and Meniere's disease.


SEQUENCE LISTING (5 sequences)

SEQ ID NO: 1 - protein, 382 aa, Homo sapiens
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRHAGGGARRAARASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLPPRPPRTARRTRRRKTRSETGGKG

SEQ ID NO: 2 - protein, 6 aa, Artificial Sequence
  Fragment - amino acids 37-42 of AF protein
  VARIANT (1)..(1): C may be replaced by S
  VARIANT (2)..(2): H may be replaced by R or K
  VARIANT (3)..(3): S may be replaced by L
  VARIANT (5)..(5): T may be replaced by A
CHSKTR

SEQ ID NO: 3 - protein, 16 aa, Artificial Sequence
  Fragment - amino acids 36-51 of AF protein
  variant (2)..(2): C may be replaced by S
  variant (3)..(3): H may be replaced by R or K
  variant (4)..(4): S may be replaced by L
  variant (6)..(6): T may be replaced by A
VCHSKTRSNPENNVGL

SEQ ID NO: 4 - protein, 7 aa, Artificial Sequence
  Fragment  - amino acids 36-42 of AF Protein
  variant (2)..(2): C may be replaced by S
  variant (3)..(3): H may be replaced by R or K
  variant (4)..(4): S may be replaced by L
  variant (6)..(6): T may be replaced by A
VCHSKTR

SEQ ID NO: 5 - protein, 8 aa, Artificial sequence
  Fragment - amino acids 35-42 of Antisecretory Factor protein
  variant (3)..(3): C may be replaced by S
  variant (4)..(4): H may be replaced by R or K
  variant (5)..(5): S may be replaced by L
  variant (7)..(7): T may be replaced by A
IVCHSKTR